(19) United States
(12) Patent Application Publication
(10) Pub. No.: US 2023/0274822 A1
(43) Pub. Date: Aug. 31, 2023

(54) OPTIMIZATION SYSTEM AND METHOD FOR MEDICAL SYSTEM, AND COMPUTER-READABLE STORAGE MEDIUM

(71) Applicant: GE Precision Healthcare LLC, Wauwatosa, WI (US)

(72) Inventor: Min Song, Shanghai (CN)

(21) Appl. No.: 18/044,722

(22) PCT Filed: Sep. 8, 2021
(86) PCT No.: PCT/US2021/049408

§ 371 Date: Mar. 9, 2023

(30) Foreign Application Priority Data
Sep. 11, 2020 (CN) 202010953962.4

(51) Int. Cl. G16H 40/20

(52) U.S. Cl. CPC G16H 40/20

(57) ABSTRACT

Provided in the present invention are an optimization method and system for a medical system, and a computer-readable storage medium. The method comprises: setting a plurality of virtual examination targets and a plurality of virtual nodes, wherein the plurality of virtual nodes separately have an initial quantity of virtual resources; controlling the plurality of virtual examination targets to sequentially pass through the plurality of virtual nodes to simulate a plurality of actual examination phases in a medical examination procedure; and in the process of simulation, determining a node to be optimized in the plurality of virtual nodes based on a current simulation result, and adjusting the quantity of virtual resources of the node to be optimized.

This application claims the benefit of and priority to Chinese Patent Application No. 202010953962.4, filed on Sep. 11, 2020, the disclosure of which is incorporated herein by reference in its entirety. The present invention relates generally to the medical field, and more specifically, to a method and a system for managing the operational efficiency of a medical institution.

### BACKGROUND

With the continuous development of modern medical technology and progress of medical systems, medical institutions such as hospitals and physical examination institutions desire to enhance operational efficiency to improve patient experience, reduce work intensity of doctors, increase the volume of patient registration, increase revenue, and the like. Although existing procedures for seeking medical attention have been greatly developed in these aspects, these developments mainly lie in factors such as improvement of medical technology and advancement of medical equipment, and less involve reasonable allocation and arrangement of medical resources. Currently, there are systems for managing assets, equipment, personnel, and so on of a medical institution. However, such systems often just perform simple resource statistics collection or equipment status monitoring, and lack advanced operational efficiency analysis and optimization capabilities.

### SUMMARY

The objective of the present invention is to overcome the above and/or other problems in the prior art.

Provided in one aspect of the present invention is an optimization method for a medical system, comprising:
setting a plurality of virtual examination targets and a plurality of virtual nodes, wherein the plurality of virtual nodes separately have an initial quantity of virtual resources;
controlling the plurality of virtual examination targets to sequentially pass through the plurality of virtual nodes to simulate a plurality of actual examination phases in a medical examination procedure; and
in the process of simulation, determining a node to be optimized in the plurality of virtual nodes based on a current simulation result, and adjusting the quantity of virtual resources of the node to be optimized.

Based on another aspect of the present invention, after the simulation ends, the quantity of virtual resources of each virtual node is recorded.

Based on another aspect of the present invention, the adjustment step comprises: adding a unit quantity of virtual resources to the node to be optimized.

Based on another aspect of the present invention, the determination step comprises: if the quantity of virtual examination targets waiting to enter a certain virtual node does not reach a minimum value, determining that the virtual node is a node to be optimized.

Based on another aspect of the present invention, the determination step comprises: if a virtual examination target is waiting to enter a certain virtual node, determining that the virtual node is a node to be optimized.

Based on another aspect of the present invention, the method further comprises:
setting maximum resource quantities for some or all of the plurality of virtual nodes; and
in the process of simulation, determining whether the quantity of virtual resources of a node to be optimized for which a maximum resource quantity is set reaches the corresponding maximum resource quantity, and if so, setting the node to be optimized as an optimized node.

Based on another aspect of the present invention, the method further comprises:
setting maximum resource quantities for some or all of the plurality of virtual nodes; and
after the simulation ends, if the quantity of virtual resources of a certain virtual node is greater than a corresponding maximum resource quantity, modifying the quantity of virtual resources of the virtual node to the set maximum resource quantity.

Based on another aspect of the present invention, the adjustment step comprises:
a parameter setting step: setting a left boundary quantity N, a right boundary quantity 2*N, and a median M, where N is the current quantity of virtual resources of the node to be optimized, and M=(N+2*N)/2;
a resource quantity setting step: setting the quantity of virtual resources of the node to be optimized to the current median M, and determining whether the quantity of virtual examination targets waiting to enter the node to be optimized reaches a minimum value;
a first determination step: if the quantity of virtual resources of the node to be optimized is M, determining whether the quantity of the virtual examination targets waiting to enter the node to be optimized reaches the minimum value;
a resource quantity increasing step: if a determination result of the first determination step is “No,” setting the quantity of virtual resources of the node to be optimized to M+1;
a second determination step: if the quantity of virtual resources of the node to be optimized is M+1, determining whether the quantity of the virtual examination targets waiting to enter the node to be optimized reaches the minimum value;
a first parameter resetting step: if a determination result of the second determination step is “No,” setting the left boundary to M, and returning to the resource quantity setting step;
a resource quantity reducing step: if a determination result of the first determination step is “Yes,” setting the quantity of virtual resources of the node to be optimized to M−1;
a third determination step: if the quantity of virtual resources of the node to be optimized is M−1, determining whether the quantity of the virtual examination targets waiting to enter the node to be optimized reaches the minimum value;
a second parameter resetting step: if a determination result of the third determination step is “Yes,” setting the right boundary to M, and returning to the resource quantity setting step;
if the determination result of the second determination step is “Yes,” determining that an optimal quantity of virtual resources of the node to be optimized is M+1; and
if the determination result of the third determination step is “No,” determining that the optimal quantity of virtual resources of the node to be optimized is M.

Based on another aspect of the present invention, the method further comprises performing one or more of the following analyses:
obtaining an average waiting time at each virtual node based on time information of each virtual examination target entering and exiting the virtual node; and
analyzing, based on an idle time length of each virtual resource, a resource utilization rate of a virtual node where the virtual resource is located.

Further provided in another aspect of the present invention is an optimization system for a medical system, comprising:
a setting module, configured to set a plurality of virtual examination targets and a plurality of virtual nodes, wherein the plurality of virtual nodes separately have an initial quantity of virtual resources;
a control module, configured to control the plurality of virtual examination targets to sequentially pass through the plurality of virtual nodes to simulate a plurality of actual examination phases in a medical examination procedure; and
a resource quantity optimization module, configured to determine, in the process of simulation, a node to be optimized in the plurality of virtual nodes based on a current simulation result, and adjust the quantity of virtual resources of the node to be optimized.

Based on another aspect of the present invention, the system further comprises a recording module configured to record the quantity of virtual resources of each virtual node after the simulation ends.

Based on another aspect of the present invention, the resource quantity optimization module is configured to add a unit quantity of virtual resources to the node to be optimized.

Based on another aspect of the present invention, if the quantity of virtual examination targets waiting to enter a certain virtual node does not reach a minimum value, it is determined that the virtual node is a node to be optimized.

Based on another aspect of the present invention, if a virtual examination target is waiting to enter a certain virtual node, it is determined that the virtual node is a node to be optimized.

Based on another aspect of the present invention, the setting module is further configured to set maximum virtual resource quantities for some or all of the plurality of virtual nodes; and
the resource quantity optimization module is further configured to: determine, in the process of simulation, whether the quantity of virtual resources of a node to be optimized for which a maximum resource quantity is set reaches the corresponding maximum resource quantity, and if so, set the node to be optimized as an optimized node.

Based on another aspect of the present invention, the setting module is further configured to: set maximum virtual resource quantities for some or all of the plurality of virtual nodes; and
the resource quantity optimization module is further configured to: modify, after the simulation ends, the quantity of virtual resources of the virtual node to the set maximum resource quantity if the quantity of virtual resources of a certain virtual node is greater than a corresponding maximum resource quantity.

Based on another aspect of the present invention, the resource quantity optimization module further comprises:
a parameter setting unit, configured to determine a left boundary quantity N, a right boundary quantity 2*N, and a median M, where N is a current quantity of virtual resources of the node to be optimized, and M=(N+2*N)/2;
a resource quantity setting unit, configured to set the quantity of virtual resources of the node to be optimized to the current median M;
a resource quantity increasing unit, configured to set the quantity of virtual resources of the node to be optimized to M+1 if the current median M does not enable the quantity of virtual examination targets waiting to enter the node to be optimized to reach a minimum value;
a quantity resetting unit, configured to reset the left boundary quantity to M, and reset the median to M=(M+2*N)/2 if M+1 does not enable the quantity of the virtual examination targets waiting to enter the node to be optimized to reach the minimum value; otherwise, set the node to be optimized as an optimized node;
a resource quantity reducing unit, configured to set the quantity of virtual resources of the node to be optimized to M−1 if the current median M enables the quantity of the virtual examination targets waiting to enter the node to be optimized to reach the minimum value, wherein
the quantity resetting unit is further configured to: reset the right boundary quantity to M, and reset the median to M=(N+M)/2 if M−1 enables the quantity of the virtual examination targets waiting to enter the node to be optimized to reach the minimum value; otherwise, set the node to be optimized as an optimized node.

Based on another aspect of the present invention, the system further comprises an analysis module configured to analyze one or both of an average waiting time and a resource utilization rate of each virtual node, wherein
the analyzing an average waiting time at each virtual node comprises: obtaining an average waiting time at each virtual node based on time information of each virtual examination target entering and exiting the virtual node; and
the analyzing a resource utilization rate of each virtual node comprises: analyzing, based on an idle time length of each virtual resource, a resource utilization rate of a virtual node where the virtual resource is located.

Based on another aspect of the present invention, the optimization system is disposed in a server, and the server is configured to communicate with one or a plurality of clients of a medical institution.

Provided in another aspect of the present invention is a computer-readable storage medium, comprising a stored computer program, wherein the method according to any one of the aforementioned aspects is performed when the computer program is run.

It should be understood that the brief description above is provided to introduce, in a simplified form, some concepts that will be further described in the Detailed Description. The brief description above is not meant to identify key or essential features of the claimed subject matter. The scope is defined uniquely by the claims that follow the detailed description. Furthermore, the claimed subject matter is not limited to implementations that solve any disadvantages noted above or in any section of the present disclosure.

Other features and aspects will become clear through the following detailed description, accompanying drawings, and claims.

### BRIEF DESCRIPTION OF THE DRAWINGS

The present invention may be better understood by means of the description of exemplary embodiments of the present invention with reference to accompanying drawings, in which:

FIG. 1 is a schematic structural diagram of an optimization system for a medical system in an embodiment of the present invention;

FIG. 2 is a module diagram of an embodiment of the optimization system 100;

FIG. 3 illustrates an example of a list of relevant information of a medical examination procedure;

FIG. 4 is a module diagram of another embodiment of the optimization system 100;

FIG. 5 illustrates an example of the plurality of virtual examination targets P₁, P₂, P₃ . . . Pₙ and the plurality of virtual nodes N₁, N₂, N₃ . . . Nₘ;

FIG. 6 illustrates an example of an optimization result of a virtual resource quantity of each virtual node obtained based on any of the aforementioned embodiments;

FIG. 7 illustrates an example of a resource allocation ratio optimization table obtained based on the optimization result;

FIG. 8 is a block diagram of an optimization system for a medical system provided in a fifth embodiment of the present invention;

FIG. 9 is a flowchart of an optimization method in an embodiment;

FIG. 10 is a flowchart of an optimization method in another embodiment;

FIG. 11 is a flowchart of another example of the adjustment step in FIG. 9;

FIG. 12 is a flowchart of an optimization method in another embodiment;

FIG. 13 is a flowchart of an optimization method in another embodiment; and

FIG. 14 is a flowchart of an optimization method in another embodiment.

### DETAILED DESCRIPTION

Specific implementations of the present invention will be described in the following. It should be noted that during the specific description of the implementations, it is impossible to describe all features of the actual implementations in detail in this description for the sake of brief description. It should be understood that in the actual implementation of any of the implementations, as in the process of any engineering project or design project, a variety of specific decisions are often made in order to achieve the developer's specific objectives and meet system-related or business-related restrictions, which will vary from one implementation to another. Moreover, it can also be understood that although the efforts made in such development process may be complex and lengthy, for those of ordinary skill in the art related to content disclosed in the present invention, some changes in design, manufacturing, production or the like based on the technical content disclosed in the present disclosure are only conventional technical means, and should not be construed as that the content of the present disclosure is insufficient.

Unless otherwise defined, the technical or scientific terms used in the claims and the description are as they are usually understood by those of ordinary skill in the art to which the present invention pertains. The terms “first,” “second,” and similar terms used in the description and claims of the patent application of the present invention do not denote any order, quantity, or importance, but are merely intended to distinguish between different constituents. “One,” “a(n),” and similar terms are not meant to be limiting, but rather denote the presence of at least one. The term “include,” “comprise,” or a similar term is intended to mean that an element or article that appears before “include” or “comprise” encompasses an element or article and equivalent elements that are listed after “include” or “comprise,” and does not exclude other elements or articles. The term “connect,” “connected,” or a similar term is not limited to a physical or mechanical connection, and is not limited to a direct or indirect connection.

First Embodiment

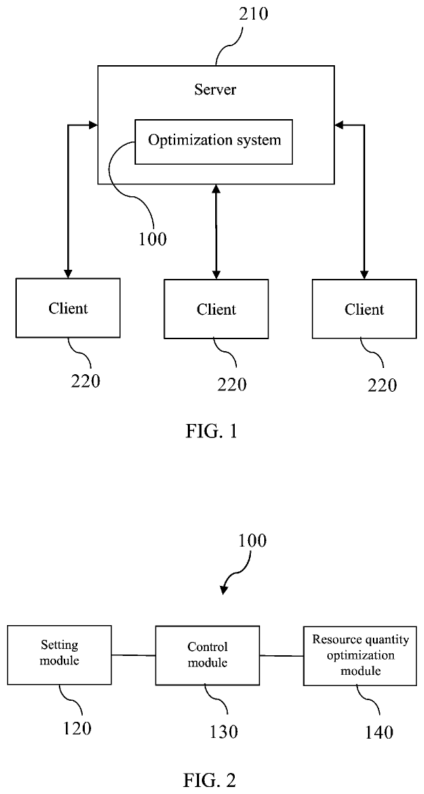

FIG. 1 is a schematic structural diagram of an optimization system for a medical system in an embodiment of the present invention. As shown in FIG. 1, the optimization system 100 may be disposed in a server 210. The server 210 may be a local server disposed in a local area network of a medical institution, a remotely disposed remote server, or a cloud computing-based cloud server. The server may be provided with a client interface to be able to communicate with one or a plurality of clients 220 of the medical institution through, for example, a local area network, a remote communication network, or a cloud network.

The aforementioned medical system may include one or a plurality of procedures for seeking medical attention or medical examination procedures of the medical institution. Such procedures may involve many aspects such as resource allocation, time allocation, and patient capacity. The embodiment of the present invention can optimize one or a plurality of aspects of the medical system, so as to improve the operational efficiency of the medical institution.

FIG. 2 is a module diagram of an embodiment of the optimization system 100. As shown in FIG. 2, the optimization system 100 includes a setting module 120, a control module 130, and a resource quantity optimization module 140.

The setting module 120 is configured to set a plurality of virtual examination targets and a plurality of virtual nodes, wherein an initial quantity of virtual resources is separately set for the plurality of virtual nodes. The control module 130 is configured to control the plurality of virtual examination targets to sequentially pass through the plurality of virtual nodes. In this way, a plurality of actual examination phases in a medical examination procedure are dynamically simulated. The resource quantity optimization module 140 is configured to determine, in the process of simulation, a node to be optimized in the plurality of virtual nodes based on a current simulation result, and adjust the quantity of virtual resources of the node to be optimized.

The aforementioned virtual nodes and virtual examination targets may be set based on the medical examination procedure. The aforementioned medical examination procedure may be a procedure that a patient needs to go through when seeking medical attention or undergoing medical examination, and may include, for example, a sequential combination of all or some of the following actual examination phases: queuing number calling, queuing number allocation, patient education, heart rate measurement, contrast agent injection, room arrangement, scan preparation, scan, image reconstruction, image post-processing, report entry, report generation, and the like.

The plurality of virtual nodes may respectively correspond to the plurality of actual examination phases. For example, the plurality of virtual nodes may be sorted in order of execution of the corresponding actual examination phases. The aforementioned virtual resources may correspond to resources such as inspection equipment, personnel allocation, and reception windows in these actual phases. The initial quantity of the virtual resources may be the minimum quantity of resources that can be provided by the medical institution for subsequent optimization of a resource allocation ratio of the plurality of actual examination phases. Specifically, the “initial quantity” may be a unit quantity “1” or a multiple thereof. That is, the allocation ratio of the virtual resources of each virtual node is set to 1:1 by the setting module 120.

Each virtual node may further have a virtual time length corresponding to an execution time of an actual examination phase. The virtual time length may be, for example, a time length starting from each virtual examination target entering the virtual node to flowing out of the virtual node in the simulation process. For example, in the actual examination phase, a patient may undergo patient education for about 3 minutes, and then the virtual time length may be set to a time length corresponding to “3 minutes” (for example, the time length may be actually 3 minutes or a shorter time length obtained by reducing the 3-minute time in a specific proportion).

In the simulation process, the control module 130 is configured to control the plurality of virtual examination targets, so that any virtual examination target passes through any virtual node by means of one of the virtual resources of the virtual node according to a virtual time length of the virtual node. For example, a time length starting from any virtual examination target flowing into any virtual node to flowing out of the virtual node by means of one virtual resource is consistent with a virtual time length of the node. The above description “a plurality of actual examination phases in a medical examination procedure are dynamically simulated” refers to any combination of the following aspects: virtual examination targets passing through each virtual node change over time; each virtual examination target flows out of/enters/remains in different virtual nodes over time; a time length for each virtual examination target to pass through each of different virtual nodes may be different; after flowing out of one virtual node, each virtual examination target may go through a certain waiting time before flowing into the next virtual node; the waiting time varies with the sequential order of a plurality of virtual examination targets; the waiting time varies with the virtual resource of the next virtual node; in the process in which the plurality of virtual examination targets sequentially pass through the plurality of virtual nodes, the control module 130 may control the state of the process, for example, end the process or interrupt the process, wherein after “ending the process,” the control module 130 may control the plurality of virtual examination targets to pass through each of the plurality of virtual nodes in sequential order again, and the “interrupting the process” is similar to suspending the process, and after “interrupting the process,” the control module 130 may control the position of each of the plurality of virtual examination targets relative to the plurality of virtual nodes to be still in the state before interruption, so as to resume the interrupted process in the state.

The resource quantity optimization module 140 may continuously search for a node to be optimized in the plurality of virtual nodes as the simulation process continues, and continuously adjust the quantity of virtual resources of each node to be optimized until the simulation ends. As the simulation process continues, when the quantity of virtual resources of a certain virtual node is adjusted to be optimal, the resource quantity optimization module 140 determines that the virtual node is not a node to be optimized or determines that the virtual node is an optimized node.

As described above, the setting module 120 may set the quantities of virtual nodes and virtual examination targets based on an actual medical examination procedure, so that the optimization system 100 can more accurately analyze the operational efficiency of the actual medical examination procedure and optimize the operational efficiency by obtaining desirable resource allocation. For example, the optimization system 100 may further include an information obtaining module, wherein the information obtaining module may be a client interface disposed on the server configured to receive relevant information of a medical examination procedure in a medical system to be optimized from the one or plurality of clients.

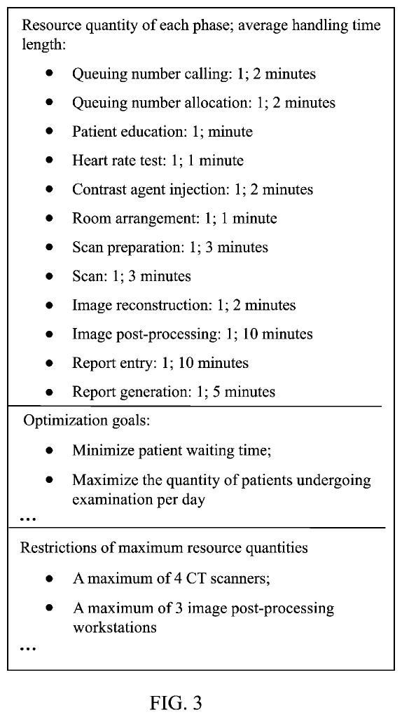

The relevant information of the medical examination procedure may include a plurality of examination phases in the procedure and a time length thereof. FIG. 3 shows an example of a list of relevant information of the medical examination procedure, wherein the list shows a plurality of sequentially arranged examination phases and average time lengths thereof. The setting module 120 may perform corresponding setting according to such relevant information. For example, if a time length for each patient to undergo a queuing number calling phase is 1 minute, the virtual time length of the corresponding virtual node is adapted to “1 minute.”

The relevant information of the medical examination procedure may further include the quantity of examination targets, wherein the quantity may be consistent with the quantity of patients handled within a specific time period (for example, a work period throughout the day or a certain part of the time period) in actual operations, or the quantity of patients that can be handled within the specific time period expected by an operations manager. The setting module 120 may determine the quantity of virtual examination targets based on the quantity of the examination targets.

The relevant information of the medical examination procedure may further include a time interval at which examination targets sequentially enter the first phase (for example, the queuing number calling phase) of the medical examination procedure. For example, in a medical examination procedure, one queuing number is called every minute. Similarly, the setting module 120 may set, based on the time interval, a time interval at which virtual examination targets sequentially enter a first virtual node.

In other embodiments, relevant information of the medical examination procedure may also be pre-stored in the server, so that the optimization system 100 can invoke the information immediately in the analysis or optimization process.

The optimization of the medical system may specifically include improvements on, for example, a resource utilization rate of the medical examination procedure, an average waiting time in each phase, and the volume of patients within a specific time, all of which can be achieved through optimization of the resource quantity.

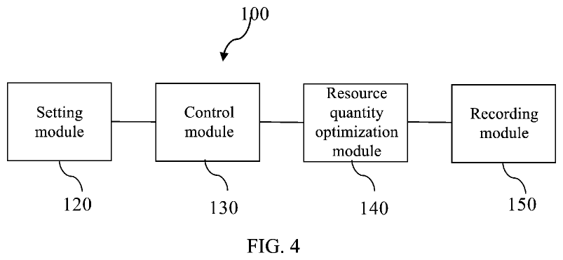

As shown in FIG. 4, the resource quantity optimization module 140 may further include a determination unit and an adjustment unit, wherein the determination unit is configured to sequentially determine whether each virtual node is a node to be optimized. Specifically, the “determination” may be performed synchronously with “simulation.” For example, the determination may be performed at any moment of the process in which the control module 130 controls the plurality of virtual examination targets to sequentially pass through the plurality of virtual nodes. The determination unit is configured to send a determination result to the adjustment unit.

If a certain virtual node is determined by the determination unit as a node to be optimized, the adjustment unit adjusts a virtual resource quantity of the virtual node, and the control module continues to perform simulation based on the adjusted virtual resource quantity until the virtual node is no longer a node to be optimized.

Specifically, if a certain virtual node is determined by the determination unit as a node to be optimized, the control module 130 stops or interrupts the aforementioned control (or simulation) process of “controlling the plurality of virtual examination targets to sequentially pass through the plurality of virtual nodes.” After the adjustment unit adjusts a virtual resource quantity of the node to be optimized, the control module 130 may restart the stopped simulation process or resume the interrupted process until the virtual node is no longer a node to be optimized.

FIG. 4 is a module diagram of another embodiment of the optimization system 100. As shown in FIG. 4, the optimization system 100 may further include a recording module 150 configured to record the quantity of virtual resources of each virtual node after the simulation ends. An optimization or adjustment scheme of a resource allocation ratio (a ratio of resource quantities) of corresponding actual examination phases can be obtained based on the recorded quantity of virtual resources of each virtual node.

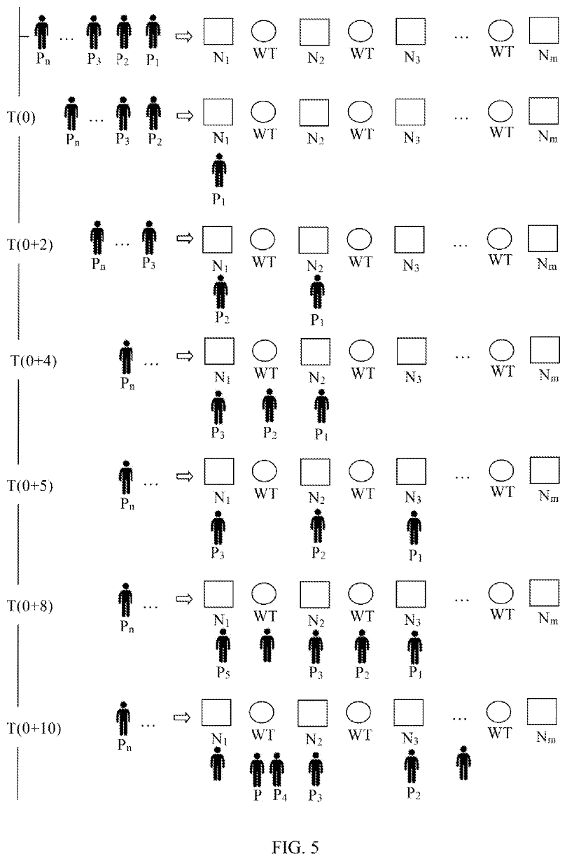

FIG. 5 illustrates an example of the plurality of virtual examination targets P₁, P₂, P₃ . . . Pₙ and the plurality of virtual nodes N₁, N₂, N₃ . . . Nₘ. An example of this embodiment is described below with reference to FIG. 1, FIG. 2, FIG. 4, and FIG. 5. For example, the setting module 120 may perform the following setting:

The plurality of virtual examination targets P₁, P₂, P₃ . . . Pₙ and the plurality of virtual nodes N₁, N₂, N₃ . . . Nₘ are set, where the subscripts n and m are both natural numbers. The virtual node N₁ corresponds to the actual queuing number calling phase, the virtual node N₂ corresponds to the actual queuing number allocation phase, the virtual node N₃ corresponds to the actual patient education phase, and so on. Virtual time lengths of the virtual node N₁, N₂, N₃ . . . are, for example, 2 minutes, 3 minutes, 5 minutes, and so on respectively. Each virtual node has 1 virtual resource, which means that the queuing number calling phase, the queuing number allocation phase, and the patient education phase are separately provided with one work window. A time interval at which virtual nodes sequentially enter the virtual node N₁ is 2 minutes.

The control module 130 controls the virtual examination target P₁ to enter the virtual node N₁ (for example, at T(0), where T represents a time point) and remain in the virtual node N₁ for 2 minutes (until T(0+2), where T(0+2) and T(0) have an interval of 2 minutes therebetween); 2 minutes after the virtual examination target P₁ enters the virtual node N₁ (until T(0+2)), the virtual examination target P₂ is controlled to enter the virtual node N₁ (at T(0+2)), and the virtual examination target P₂ remains in the virtual node N₁ for 2 minutes (until T(0+4)); 2 minutes after the virtual examination target P₂ enters the virtual node (T(0+4)), the virtual examination target P₃ is controlled to enter the virtual node N₁ (at T(0+4)), and remain in the virtual node N₁ for 2 minutes until T(0+6), and so on.

The control module 130 controls the virtual examination target P₁ to flow out of the virtual node N₁ (at T(0+2)) and immediately enter the virtual node N₂, remain in the virtual node N₂ for 3 minutes and then directly enter the virtual node N₃ (at T(0+5)), and remain in the virtual node N₃ for 5 minutes (until T(0+10)), then flow out and immediately enter the next node; the virtual examination target P₂ flows out of the virtual node N₁ (at T(0+4)), waits 1 minute and then enters the virtual node N₂ (at T(0+5)); the virtual examination target P₂ remains in the virtual node N₂ for 3 minutes (until T(0+8)), needs to wait 2 minutes and then enters the virtual node N₃ (at T(0+10)), and remains in the virtual node N₃ for 5 minutes (until T(0+15)); the virtual examination target P₃ flows out of the virtual node N₁ (at T(0+6)), needs to wait 2 minutes and then enters the virtual node N₂ (at T(0+8)); the virtual examination target P₃ remains in the virtual node N₂ for 3 minutes (until T(0+11)), needs to wait 4 minutes and then enters the virtual node N₃ (at T(0+15)), and so on.

According to the current quantity of resources, since virtual time lengths at different virtual nodes are different, a node having a greater virtual time length tends to have a virtual examination target waiting for entry. For example, at the moment T(0+4), the virtual node N₂ has the virtual examination target P₂ waiting for entry; at the moment T(0+8), the virtual node N₂ has at least the virtual examination target P₄ waiting for entry, and the virtual node N₃ has at least the virtual examination target _{P}2 waiting for entry; and at the moment T(0+10), the virtual node N₂ has the virtual examination targets P₄ and P5 waiting for entry.

For ease of description, in FIG. 5, a waiting node WT is set between adjacent virtual nodes. When one or a plurality of virtual examination targets flowing out of one virtual node cannot flow into the next node immediately, the one or plurality of virtual examination targets are arranged in sequential order at a waiting node between the two virtual nodes until they can flow into the next virtual node.

In a specific embodiment, each virtual node can exactly have no virtual examination target waiting for entry by means of “optimization while simulation,” wherein the term “exactly” means that a minimum quantity of virtual resources are used to enable the quantity of virtual examination targets waiting before each virtual node to reach a minimum value; for example, there is no virtual examination target waiting before each virtual node.

For example, the determination unit may determine whether a virtual node has a virtual examination target waiting for entry, and if so, determine the virtual node as a node to be optimized. For example, when the simulation process proceeds to the time point T(0+4) and it is found that the virtual node N₂ has at least the virtual examination target P₂ waiting for entry, the virtual node N₂ is temporarily determined as a node to be optimized. At this time, the control module 130 may temporarily interrupt or end the simulation process, and adjust the quantity of virtual resources of the virtual node (or the node to be optimized) N₂ through the optimization module 150, for example, adjust the quantity of virtual resources of the virtual node N₂ from the initial value “1” to 2. After the optimization module 150 adjusts the quantity of virtual resources of the virtual node N₂, the control module 130 may restart the ended simulation process or resume the interrupted simulation process.

In the simulation process that has been performed again, since the virtual node N₂ has two virtual resources capable of running in parallel, after flowing out of the virtual node N₁ (at T(0+4)), the virtual examination target P₂ can directly enter the virtual node N₂ without waiting. At this time, the determination module 140 determines that the virtual node N₂ is temporarily not a node to be optimized, therefore determine that the virtual node N₂ is no longer a node to be optimized, or further temporarily set the virtual node N₂ as an optimized node.

As another example, when the control process proceeds to the time point T(0+8) and it is found that the virtual node N₃ has at least the virtual examination target P₂ waiting for entry, the virtual node N₃ is temporarily determined as a node to be optimized. At this time, the control module 130 interrupts or ends the simulation process, and adjusts the quantity of virtual resources of the virtual node (or the node to be optimized) N₃ through the optimization module 150, for example, adjusts the quantity of virtual resources of the virtual node N₃ from the initial value “1” to 2. After the quantity of virtual resources of the virtual node N₂ is adjusted, the control module 130 may restart the ended simulation process or resume the interrupted simulation process.

In the simulation process that has been performed again, since the virtual node N₃ has two virtual resources capable of running in parallel, after flowing out of the virtual node N₂ (at T(0+7)), the virtual examination target P₂ can directly enter the virtual node N₃ without waiting.

At this time, the determination unit 141 determines that the virtual node N₂ is temporarily not a node to be optimized, and therefore temporarily set the virtual node N₂ as an optimized node.

If an optimized node has a virtual examination target waiting again in the subsequent simulation process, the aforementioned steps are repeated to adjust the quantity of virtual resources at the node again, so that the quantity of virtual resources of each virtual node can be dynamically adjusted based on the current simulation result. Therefore, the recording module 150 is specifically configured to record the quantity of virtual resources of each virtual node after the simulation process completely ends. For example, when the last virtual examination target in all virtual examination targets flows out of the last virtual node, it can be determined that the simulation process completely ends, and at this time, the quantities of resources of all the virtual nodes (optimized nodes) are recorded through the recording module 150.

A ratio of the quantities of resources of the optimized nodes may be provided as an optimized resource allocation ratio to the client, or various optimization indices of the operational efficiency are calculated based on the optimized resource allocation ratio. For example, the optimized resource utilization rate, the optimized average patient waiting time, the optimized patient handling capacity, the optimized total patient handling duration, and the like are calculated. This will be described in detail below.

In this embodiment, the resource quantity optimization module may add a unit quantity of virtual resources to the current node to be optimized, so that the quantity of the virtual resources can be dynamically adjusted. For example, every time the determination unit determines that a certain virtual node is a node to be optimized, the adjustment unit adds a unit quantity of virtual resources to the node to be optimized; for example, the quantity increases from the initial quantity “one” to “two”, and if the node is determined as a node to be optimized again, the quantity increases from “two” to “three,” and so on. In this way, the problem of unsatisfactory optimization result caused by allocating excess virtual resources for reducing queues can be avoided, and the minimum quantity that can avoid waiting can be found in a simple processing manner.

As described above, after the resource quantity optimization module 140 adjusts the quantity of virtual resources of the node to be optimized, the control module 130 may restart the stopped simulation process, that is, control the virtual examination targets P₁, P₂, P₃ . . . Pₙ again to sequentially pass through N₁, N₂, N₃ . . . Nₘ. In this way, repeated recording of the simulation process can be avoided. For example, recording which virtual examination target or which virtual node corresponds to the current simulation process is avoided.

However, in order to reduce the number of repetitions of the simulation and shorten the simulation time, after the quantity of virtual resources of the node to be optimized is adjusted, the control module 130 may continue to start the interrupted simulation process, that is, continue to perform the simulation process before interruption. For example, when the simulation proceeds to the time point T(0+8), the determination unit finds that the virtual node N₃ has a virtual examination target P₂ waiting for entry, and thus determines the virtual node N₃ as a node to be optimized, and the simulation process is interrupted at this time; the adjustment unit adjusts the quantity of virtual resources of the virtual node N₃ from 1 to 2, and causes the virtual examination target P₂ to enter the virtual node N₃ at the time point T(0+7); and the interrupted simulation process is resumed without requiring the virtual examination target P₁ to pass through the virtual nodes N₁, N₂, and N₃ again, or requiring the virtual examination target P₂ to pass through the virtual nodes N₁ and N₂ again. If the virtual node N₃ still has a virtual examination target P₃ waiting while this simulation process continues to proceed, the process is interrupted again, the quantity of resources is adjusted, and the virtual examination target P₃ is made to enter the virtual node N₃ at the time point T(0+8), so as to continue to perform the simulation process that has been interrupted again.

### Second Embodiment

Provided in the second embodiment of the present invention is an optimization system for a medical system that is similar to the first embodiment in structure, principle, and so on, except that in order to reduce the number of repetitions of determination, simulation, and optimization or simplify the processing manner of the corresponding modules, when adjusting the quantity of virtual resources of a node to be optimized, the resource quantity optimization module 140 assigns an assumed quantity of virtual resources to the node in a specific manner rather than adjusting one unit quantity at a time, which is particularly applicable to a node having a large amount of resources.

In some examples, the above assumption is not an arbitrary assumption; instead, first, a large quantity range is set based on the current quantity, and the quantity range is continuously adjusted according to the determination result of “whether the quantity of virtual examination targets waiting to enter the node to be optimized reaches a minimum value,” until an optimal quantity of resources is obtained. That is, the finally determined quantity of resources meets the optimization goal without causing resource redundancy.

For example, when it is found, based on the current simulation result, that virtual examination targets waiting before the node to be optimized are at the minimum quantity or do not exist, the resource quantity optimization module 140 determines that the quantity of virtual resources of the node to be optimized has met the optimization goal.

However, the quantity of virtual resources may be excessively large to cause redundancy. Therefore, it is further necessary to determine whether the quantity is an optimal quantity.

The aforementioned optimization goal may include at least one of the following items: maximizing the quantity of examination targets completing the medical examination procedure per unit time; minimizing the average waiting time in each actual examination phase (as illustrated in FIG. 3); and minimizing the time for a specific examination target to complete the medical examination procedure. The optimization goals all can be achieved by minimizing the average waiting time in each phase. For example, the quantity of virtual examination targets waiting to enter each virtual node can be minimized by optimizing the resource quantity.

Based on the above objective, the resource quantity optimization unit may include a determination unit, a parameter setting unit, a resource quantity setting unit, a resource quantity increasing unit, a parameter resetting unit, and a resource quantity reducing unit.

The parameter setting unit is configured to determine a left boundary quantity N, a right boundary quantity 2*N, and a median M, where N is a current quantity of virtual resources of the node to be optimized, and M=(N+2*N)/2. In an embodiment, if (2+2*N)/2 is not an integer, the result is rounded and assigned to M. For example, if N is 5, (2+2*N)=7.5; at this time, the figure is rounded off to obtain an integer 8, and then the median M=8.

The resource quantity setting unit is configured to set the quantity of virtual resources of the node to be optimized to the current median M.

The determination unit is configured to determine whether the quantity of virtual examination targets waiting to enter the node to be optimized reaches a minimum value when the quantity of virtual resources of the node to be optimized is adjusted (for example, from the initial quantity to the current median M).

The resource quantity increasing unit is configured to set the quantity of virtual resources of the node to be optimized to M+1 if the current median M does not enable the quantity of the virtual examination targets waiting to enter the node to be optimized to reach the minimum value.

The quantity resetting unit is configured to reset the left boundary quantity to M (for example, such that N=M), and reset the median to M=(M+2*N)/2 if M+1 does not enable the quantity of the virtual examination targets waiting to enter the node to be optimized to reach the minimum value. At this time, the quantity of virtual resources of the node to be optimized is also readjusted to M=(M+2*N)/2 by the resource quantity setting unit.

The resource quantity reducing unit is configured to set the quantity of virtual resources of the node to be optimized to M−1 if the current median M enables the quantity of the virtual examination targets waiting to enter the node to be optimized to reach the minimum value.

The quantity resetting unit is further configured to: reset the right boundary quantity to M (for example, such that 2*N=M) and reset the median to M=(N+M)/2 if M−1 enables the quantity of the virtual examination targets waiting to enter the node to be optimized to reach the minimum value.

If M+1 or M−1 enables the quantity of the virtual examination targets waiting to enter the node to be optimized to reach the minimum value, it indicates that M+1 or M−1 meets the optimization goal, and the determination module determines that the node is no longer a node to be optimized (or determines that the node is an optimized node).

Optionally, before setting the quantity of virtual resources of the node to be optimized to the median M for the first time, the resource quantity setting unit sets the quantity of virtual resources of the node to be optimized to the right boundary quantity 2*N. The determination unit is further configured to: determine whether the quantity of the virtual examination targets waiting to enter the node to be optimized reaches the minimum value if the quantity of virtual resources of the node to be optimized is set to the right boundary quantity 2*N, and if so, set the quantity of the virtual resources to the median M, and if not, the current quantity of the virtual resources (namely, the left boundary quantity) is 2*N, and the right boundary quantity is 2*2*N.

Detailed description is provided below by way of example:

If a certain virtual node is determined as a node to be optimized for the first time, the current quantity of virtual resources of the node is 2*1=2, and if the quantity of virtual resources is 2, the node still needs to be optimized, and the quantity of virtual resources is set to 2*2, namely, 4. If the node still needs to be optimized, the quantity of virtual resources is set to 2*4, namely, 8. If the node still needs to be optimized, the quantity of virtual resources is set to 2*8, namely, 16. If there is no waiting before the node when the quantity is 16, a left boundary is set to 8, a right boundary is set to 16, and a median is set to 12 ((8+16)/2).

When the simulation process is performed based on the quantity “12,” it is found that the quantity of virtual examination targets waiting before the node to be optimized is at the minimum or zero, which indicates that the quantity has met the optimization goal. However, the node to be optimized still cannot be determined as an optimized node yet. It needs to first assume that the quantity “12” has redundancy; that is, the quantity “12” is not the optimal quantity that meets the optimization goal. At this time, the quantity of virtual resources of the node to be optimized is set to “11 (M−1).” If the simulation result still shows that there is no waiting before the node when the quantity of virtual resources is 11, the right boundary is set to 12, the median is recalculated as 10 ((8+12)/2), and the optimal quantity of virtual resources of the node is determined based on the recalculated median. If the simulation result shows that there is waiting before the node when the quantity of virtual resources is 11, the quantity of virtual resources of the node is set to 12 (an optimal result), and it is determined that the node is no longer a node to be optimized.

When the simulation process is performed based on the quantity “12,” it is found that a virtual examination target is waiting before the node to be optimized, which indicates that the quantity does not meet the optimization goal. At this time, the quantity of virtual resources of the node to be optimized is set to “13 (M+1).” If the simulation result shows that there is still waiting before the node when the quantity of virtual resources is 13, the left boundary is set to 12, the median is recalculated as 14 ((12+16)/2), and the optimal quantity of virtual resources of the node is determined based on the recalculated median. If the simulation result shows that there is no waiting before the node when the quantity of virtual resources is 13, it is considered that the optimal quantity of virtual resources of the node is 13, and it is determined that the node is no longer a node to be optimized.

In an embodiment, when the calculated median M of the left boundary quantity N and the right boundary quantity 2*N is not an integer, the median is rounded down to the nearest integer or rounded up to the nearest integer.

In some application scenarios with a large amount of resources, rapid optimization of the medical system can be realized through this embodiment.

### Third Embodiment

Provided in the third embodiment of the present invention is an optimization system for a medical system that is similar to the first embodiment or the second embodiment in structure, principle, and so on, except that:

the setting module 120 is further configured to set maximum resource quantities for some or all of the plurality of virtual nodes. For example, the relevant information of the medical examination procedure inputted through the client or pre-stored in the server includes a maximum resource quantity of one or a plurality of phases or other information related to the maximum resource quantity, for example, a total budget of the phase. Accordingly, the setting module 120 may set the restriction based on the “relevant information of the medical examination procedure.”

Such a restriction may result in inability to zero out the quantity of virtual examination targets waiting before the corresponding virtual node due to insufficient virtual resources. However, the objective of this embodiment may be to optimize, when the aforementioned restriction is set for a certain virtual node, the quantity of virtual examination targets waiting before the virtual node to a minimum value, so as to optimize the operational efficiency under limited resource conditions.

As described in the above embodiment, in the process of simulation, the resource quantity optimization module 140 may determine whether the quantity of virtual resources of a node to be optimized for which a maximum resource quantity is set reaches the corresponding maximum resource quantity, and if so, set the node to be optimized as an optimized node. For example, in the process of simulation, the determination unit 141 may be configured to sequentially determine whether the quantity of virtual examination targets waiting for entry before each virtual node reaches a minimum value (the minimum value may be 0), and if the quantity of virtual examination targets waiting for entry before a certain virtual node does not reach the minimum value, determine the virtual node as a node to be optimized.

If a certain virtual node is determined as a node to be optimized by the determination unit, and a maximum resource quantity is set for the node to be optimized, the determination unit may further determine whether the current quantity of virtual resources of the node reaches the maximum resource quantity. If so, the node to be optimized is set as an optimized node, and the control module 130 may continue to perform the simulation process or restart the simulation process after the node to be optimized becomes an optimized node. If the quantity of virtual resources of the node to be optimized for which the maximum resource quantity is set does not reach the corresponding maximum resource quantity, the node to be optimized is optimized based on any of the aforementioned embodiments.

Therefore, even if the quantity of virtual examination targets waiting before a certain virtual node still has room for optimization (for example, not zeroed) so that the virtual node is determined as a node to be optimized, the node can be changed to an optimized node by using a limited maximum resource quantity to avoid redundant simulation or optimization processing.

### Fourth Embodiment

Provided in the fourth embodiment of the present invention is an optimization system for a medical system that is similar to the third embodiment in structure, principle, and so on, except that:

the setting module 120 is further configured to set maximum resource quantities for some or all of the plurality of virtual nodes.

After the simulation ends, the quantity of virtual resources of each virtual node is determined and recorded based on the first embodiment or the second embodiment. If the quantity of virtual resources of a certain virtual node is greater than a corresponding maximum resource quantity, the quantity of virtual resources of the virtual node is modified to the set maximum resource quantity.

For example, if the quantity of virtual resources of a certain virtual node is 5 in a preferred optimization scheme, but the setting module 120 has set the maximum resource quantity of the node to 3, after the entire simulation and optimization process ends, the quantity of virtual resources of the node is directly modified to 3, so that the restriction condition can be met and the operational efficiency can be optimized.

### Fifth Embodiment

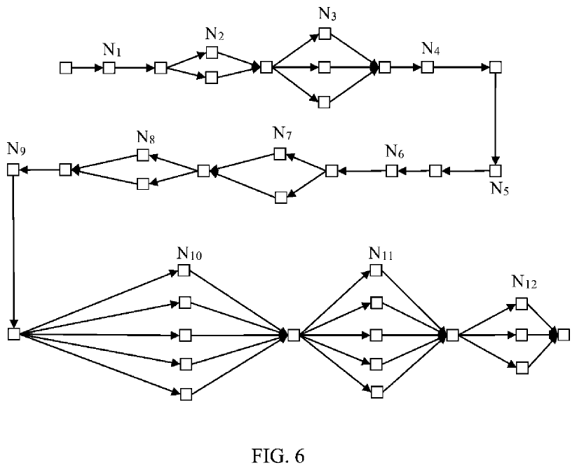

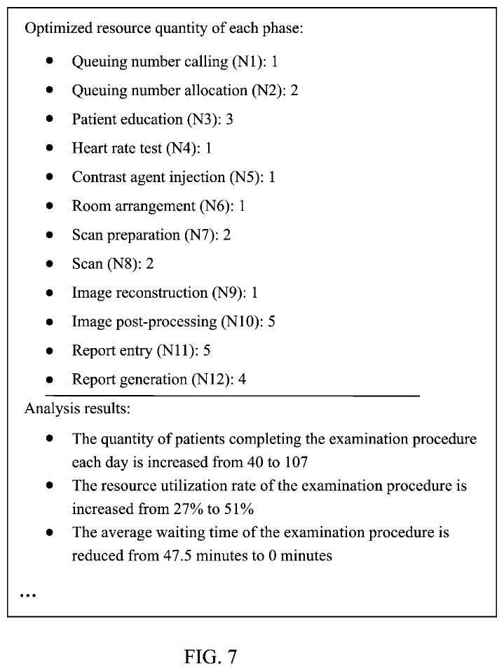

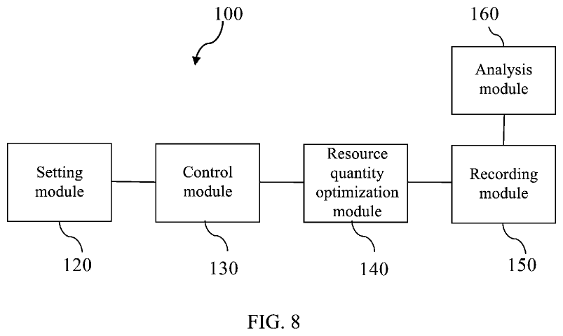

An optimized resource allocation ratio of each virtual node is obtained based on any of the aforementioned embodiments. FIG. 6 illustrates an example of an optimization result of a virtual resource quantity of each virtual node obtained based on any of the aforementioned embodiments. FIG. 7 illustrates an example of a table of an optimized resource allocation ratio obtained based on the optimization result. The optimized resource allocation ratio may be sent to the client. FIG. 8 is a block diagram of an optimization system for a medical system provided in the fifth embodiment of the present invention. As shown in FIG. 8, the optimization system may further include an analysis module 160, which may be configured to analyze a current working efficiency of the medical system based on a current resource allocation ratio in the medical examination procedure, or may be configured to analyze an optimized working efficiency of the medical system based on an optimized resource allocation ratio. The current working efficiency and the optimized working efficiency may further be compared and analyzed.

In an example, the analyzing the working efficiency of the medical system may include: analyzing a resource utilization rate of each virtual node. Specifically, based on an idle time length of each virtual resource, a resource utilization rate of a virtual node where the virtual resource is located is analyzed. For example, a ratio of an idle time of virtual resources at each virtual node to a total time length of the virtual node within a time period from the first virtual examination target entering the first virtual node to the last virtual examination target flowing out of the last virtual node may be calculated. The total time length of the virtual node may be a time length from the first virtual examination target entering the virtual node to the last virtual examination target flowing out of the virtual node. A resource utilization rate of the entire procedure may further be obtained based on resource utilization rates of all the virtual nodes (for example, the resource utilization rates of the plurality of nodes are averaged).

In another example, the analyzing the working efficiency of the medical system may further include: analyzing an average waiting time at each virtual node. Specifically, an average waiting time at each virtual node is obtained based on time information of each virtual examination target entering and exiting the virtual node. For example, an average value of time lengths for which virtual examination targets wait before each virtual node is calculated. An average waiting time of the entire procedure may further be obtained based on average waiting time at all the virtual nodes (for example, the average waiting times of the plurality of nodes are summed).

In another example, the quantity of patients that can be handled within a specific time and the length of time required for handling a certain quantity of patients may also be analyzed based on the average waiting time at each virtual node.

Any of the aforementioned analysis results of the analysis module 160 may be sent to the client. As shown in FIG. 8, the analysis result may be sent to the client along with the optimized resource allocation ratio as an optimization result.

The analysis module 160 may send the optimization result to the client through, for example, the client interface on the server.

In the description of various embodiments of the optimization system of the present invention, the “modules” may be implemented by software, hardware, or a combination of software and hardware. These “modules” may be implemented as computer program modules.

### Sixth Embodiment

Further provided in the present invention may be a plurality of embodiments of an optimization method for a medical system. The optimization method and the aforementioned optimization system have a general inventive concept. Specifically, the optimization system in the embodiments of the present invention may be implemented by the optimization system in any of the aforementioned embodiments.

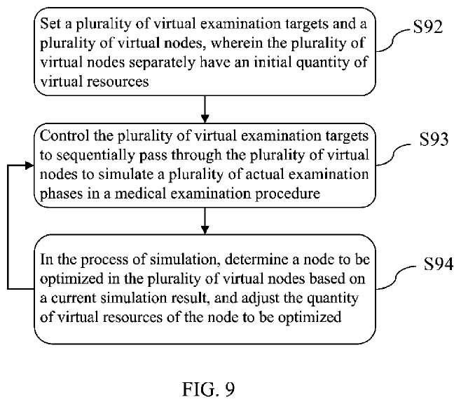

FIG. 9 is a flowchart of an optimization method in an embodiment. As shown in FIG. 9, the method includes the following steps:

Step S92: set a plurality of virtual examination targets and a plurality of virtual nodes, wherein the plurality of virtual nodes separately have an initial quantity of virtual resources.

Step S93: control the plurality of virtual examination targets to sequentially pass through the plurality of virtual nodes to simulate a plurality of actual examination phases in a medical examination procedure.

Step S94: in the process of simulation, determine a node to be optimized in the plurality of virtual nodes based on a current simulation result, and adjust the quantity of virtual resources of the node to be optimized.

### Seventh Embodiment

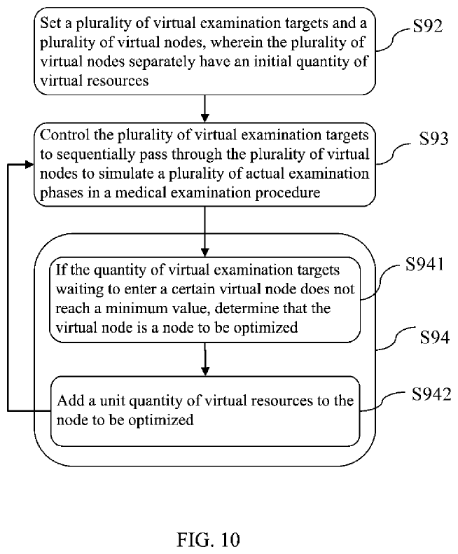

FIG. 10 is a flowchart of an optimization method in another embodiment, wherein a “determination” step S941 and an “adjustment” step S942 in step S94 are further illustrated. As shown in FIG. 10, the “determination” may include: if the quantity of virtual examination targets waiting to enter a certain virtual node does not reach a minimum value, determining that the virtual node is a node to be optimized. In a specific example, the minimum value is 0; that is, if a virtual examination target is waiting to enter a certain virtual node, it is determined that the virtual node is a node to be optimized.

In an example, the “adjustment” may include: adding a unit quantity of virtual resources to the node to be optimized, for example, adding one virtual resource at a time, until it is determined that the node is no longer a node to be optimized.

### Eighth Embodiment

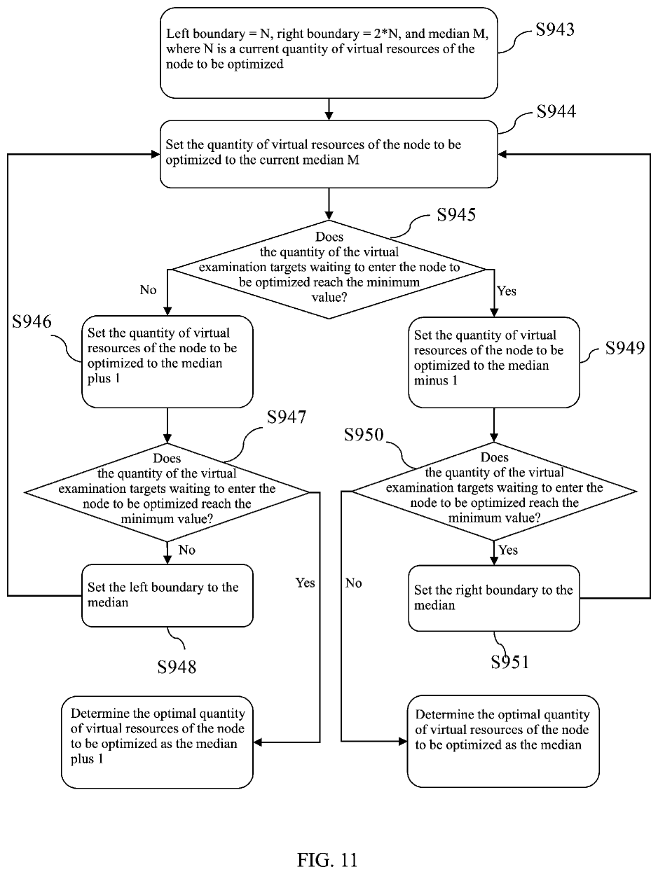

FIG. 11 is a flowchart of another example of step S94, which includes:
a parameter setting step S943: set a left boundary quantity N, a right boundary quantity 2*N, and a median M, where N is a current quantity of virtual resources of the node to be optimized, and M=(N+2*N)/2;
a resource quantity setting step S944: set the quantity of virtual resources of the node to be optimized to the current median M;
a first determination step S945: if the quantity of virtual resources of the node to be optimized is M, determine whether the quantity of virtual examination targets waiting to enter the node to be optimized reaches a minimum value;
a resource quantity increasing step S946: if the quantity M does not enable the quantity of the virtual examination targets waiting to enter the node to be optimized to reach the minimum value (the determination result of the first determination step S945 is “No”), set the quantity of virtual resources of the node to be optimized to M+1;
a second determination step S947: if the quantity of virtual resources of the node to be optimized is M+1, determining whether the quantity of the virtual examination targets waiting to enter the node to be optimized reaches the minimum value;
a first parameter resetting step S948: if M+1 does not enable the quantity of the virtual examination targets waiting to enter the node to be optimized to reach the minimum value (the determination result of the second determination step S947 is “No”), set the left boundary to M, and return to the resource quantity setting step S945;
a resource quantity reducing step S949: if the quantity M enables the quantity of the virtual examination targets waiting to enter the node to be optimized to reach the minimum value (the determination result of the first determination step S945 is “Yes”), set the quantity of virtual resources of the node to be optimized to M−1;
a third determination step S950: if the quantity of virtual resources of the node to be optimized is M−1, determine whether the quantity of the virtual examination targets waiting to enter the node to be optimized reaches the minimum value; and
a second parameter resetting step S951: if M−1 enables the quantity of the virtual examination targets waiting to enter the node to be optimized to reach the minimum value (the determination result of the third determination step S950 is “Yes”), set the right boundary to M, and return to the resource quantity setting step S944.

If M+1 enables the quantity of the virtual examination targets waiting to enter the node to be optimized to reach the minimum value (the determination result of the second determination step S947 is “Yes”), the optimal quantity of virtual resources of the node to be optimized is determined as M+1, and the procedure ends;
if M−1 does not enable the quantity of the virtual examination targets waiting to enter the node to be optimized to reach the minimum value (the determination result of the third determination step S950 is “No”), the optimal quantity of virtual resources of the node to be optimized is determined as M, and the procedure ends.

### Ninth Embodiment

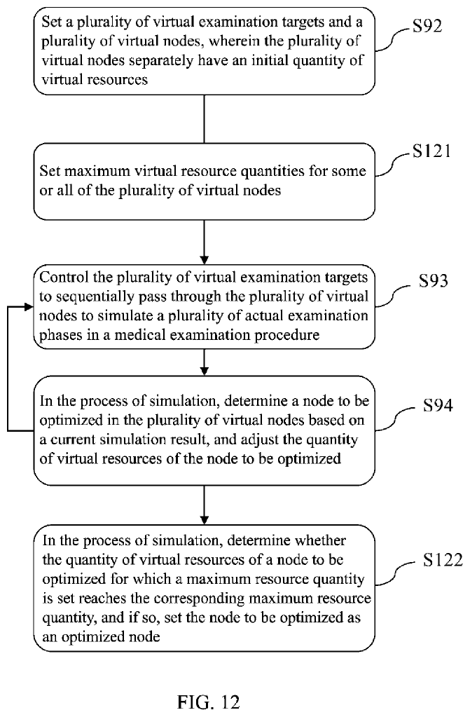

FIG. 12 is a flowchart of an optimization method in another embodiment, wherein the steps in FIG. 9 are shown, and step S121 and step S122 are further shown.

In step S121, maximum virtual resource quantities are set for some or all of the plurality of virtual nodes;
in step S122, in the process of simulation, it is determined whether the quantity of virtual resources of a node to be optimized for which a maximum resource quantity is set reaches the corresponding maximum resource quantity, and if so, the node to be optimized is set as an optimized node.

### Tenth Embodiment

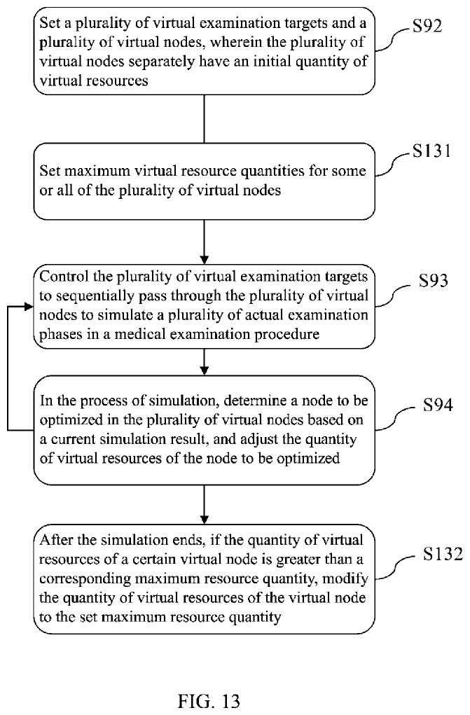

FIG. 13 is a flowchart of an optimization method in another embodiment, wherein the steps in FIG. 9 are shown, and step S131 and step S132 are further shown.

In step S131, maximum virtual resource quantities are set for some or all of the plurality of virtual nodes;
in step S132, after the simulation ends, if the quantity of virtual resources of a certain virtual node is greater than a corresponding maximum resource quantity, the quantity of virtual resources of the virtual node is modified to the set maximum resource quantity.

### Eleventh Embodiment

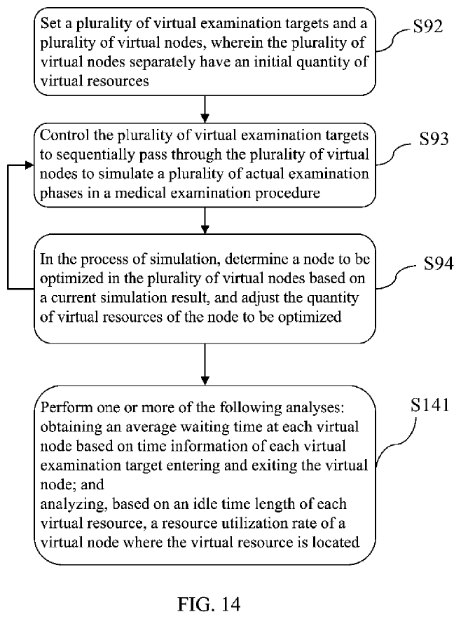

FIG. 14 is a flowchart of an optimization method in another embodiment, wherein the steps in FIG. 9 are shown, and an analysis step S141 is further shown. The analysis step S141 includes: performing one or more of the following analyses:
obtaining an average waiting time at each virtual node based on time information of each virtual examination target entering and exiting the virtual node; and
analyzing, based on an idle time length of each virtual resource, a resource utilization rate of a virtual node where the virtual resource is located.

Although the steps of the optimization method according to the specific embodiments of the present invention are shown as functional blocks, the order of the functional blocks and the separation of actions between the functional blocks shown in the drawings are not intended to be limiting. For example, the functional blocks may be performed in a different order, and an action associated with one functional block may be combined with one or a plurality of other functional blocks or may be subdivided into a plurality of functional blocks.

### Twelfth Embodiment

Further provided in an embodiment of the present invention is a computer-readable storage medium, including a stored computer program, wherein the optimization method in any of the aforementioned embodiments is performed when the computer program is run. For example, the computer program may include a plurality of stored computer program modules, and the plurality of computer program modules may include the plurality of modules described in the embodiments of the aforementioned optimization system. The storage medium may include, for example, a ROM, a floppy disk, a hard disk, an optical disk, a magneto-optical disk, a CD-ROM, or a non-volatile memory card, and may be disposed in the aforementioned server.

While the present invention has been described in detail with reference to the specific embodiments, it will be understood by those skilled in the art that many modifications and variations can be made to the present invention. Therefore, it should be understood that the claims are intended to cover all such modifications and variations within the true spirit and scope of the present invention. Some exemplary embodiments have been described above. However, it should be understood that various modifications can be made. For example, if the described techniques are performed in a different order and/or if the components of the described system, architecture, device, or circuit are combined in other manners and/or replaced or supplemented with additional components or equivalents thereof, a suitable result can be achieved. Accordingly, other implementations also fall within the protection scope of the claims.

1. A method for a medical system, comprising:
setting a plurality of virtual examination targets and a plurality of virtual nodes, wherein the plurality of virtual nodes separately have an initial quantity of virtual resources; and
controlling the plurality of virtual examination targets to sequentially pass through the plurality of virtual nodes to simulate a plurality of actual examination phases in a medical examination procedure,
wherein the simulating comprises determining a node to be optimized in the plurality of virtual nodes based on a current simulation result, and adjusting the quantity of virtual resources of the node to be optimized.

2. The method according to claim 1, further comprising: after the simulation ends, recording the quantity of virtual resources of each virtual node.

3. The method according to claim 1, wherein the adjusting comprises: adding a unit quantity of virtual resources to the node to be optimized.

4. The method according to claim 1, wherein the determining comprises: if the quantity of virtual examination targets waiting to enter a certain virtual node does not reach a minimum value, determining that the virtual node is a node to be optimized.

5. The method according to claim 4, wherein the determining comprises: if a virtual examination target is waiting to enter a certain virtual node, determining that the virtual node is a node to be optimized.

6. The method according to claim 4, further comprising:
setting maximum resource quantities for some or all of the plurality of virtual nodes; and
in the process of simulation, determining whether the quantity of virtual resources of a node to be optimized for which a maximum resource quantity is set reaches the corresponding maximum resource quantity, and if so, setting the node to be optimized as an optimized node.

7. The method according to claim 1, further comprising:
setting maximum resource quantities for some or all of the plurality of virtual nodes; and
after the simulation ends, if the quantity of virtual resources of a certain virtual node is greater than a corresponding maximum resource quantity, modifying the quantity of virtual resources of the virtual node to the set maximum resource quantity.

8. The method according to claim 1, wherein the adjusting comprises:
a parameter setting step: setting a left boundary quantity N, a right boundary quantity 2*N, and a median M, where N is the current quantity of virtual resources of the node to be optimized, and M=(N+2*N)/2;
a resource quantity setting step: setting the quantity of virtual resources of the node to be optimized to the current median M, and determining whether the quantity of virtual examination targets waiting to enter the node to be optimized reaches a minimum value;
a first determination step: if the quantity of virtual resources of the node to be optimized is M, determining whether the quantity of the virtual examination targets waiting to enter the node to be optimized reaches the minimum value;
a resource quantity increasing step: if a determination result of the first determination step is “No,” setting the quantity of virtual resources of the node to be optimized to M+1;
a second determination step: if the quantity of virtual resources of the node to be optimized is M+1, determining whether the quantity of the virtual examination targets waiting to enter the node to be optimized reaches the minimum value;
a first parameter resetting step: if a determination result of the second determination step is “No,” setting the left boundary to M, and returning to the resource quantity setting step;
a resource quantity reducing step: if a determination result of the first determination step is “Yes,” setting the quantity of virtual resources of the node to be optimized to M−1;
a third determination step: if the quantity of virtual resources of the node to be optimized is M−1, determining whether the quantity of the virtual examination targets waiting to enter the node to be optimized reaches the minimum value;
a second parameter resetting step: if a determination result of the third determination step is “Yes,” setting the right boundary to M, and returning to the resource quantity setting step;
if the determination result of the second determination step is “Yes,” determining that an optimal quantity of virtual resources of the node to be optimized is M+1; and
if the determination result of the third determination step is “No,” determining that the optimal quantity of virtual resources of the node to be optimized is M.

9. The method according to claim 1, further comprising performing one or more of the following:
obtaining an average waiting time at each virtual node based on time information of each virtual examination target entering and exiting the virtual node; and
analyzing, based on an idle time length of each virtual resource, a resource utilization rate of a virtual node where the virtual resource is located.

10. An optimization system for a medical system, comprising:
a setting module, configured to set a plurality of virtual examination targets and a plurality of virtual nodes, wherein the plurality of virtual nodes separately have an initial quantity of virtual resources;
a control module, configured to control the plurality of virtual examination targets to sequentially pass through the plurality of virtual nodes to simulate a plurality of actual examination phases in a medical examination procedure; and
a resource quantity optimization module, configured to determine, in the process of simulation, a node to be optimized in the plurality of virtual nodes based on a current simulation result, and adjust the quantity of virtual resources of the node to be optimized.

11. The optimization system according to claim 10, further comprising a recording module configured to record the quantity of virtual resources of each virtual node after the simulation ends.

12. The optimization system according to claim 10, wherein the resource quantity optimization module is configured to add a unit quantity of virtual resources to the node to be optimized.

13. The optimization system according to claim 10, wherein if the quantity of virtual examination targets waiting to enter a certain virtual node does not reach a minimum value, it is determined that the virtual node is the node to be optimized.

14. The optimization system according to claim 13, wherein if a virtual examination target is waiting to enter a certain virtual node, it is determined that the virtual node is the node to be optimized.

15. The optimization system according to claim 13, wherein
the setting module is further configured to set maximum virtual resource quantities for some or all of the plurality of virtual nodes; and
the resource quantity optimization module is further configured to: determine, in the process of simulation, whether the quantity of virtual resources of a node to be optimized for which a maximum resource quantity is set reaches the corresponding maximum resource quantity, and if so, set the node to be optimized as an optimized node.

16. The optimization system according to claim 10, wherein
the setting module is further configured to set maximum virtual resource quantities for some or all of the plurality of virtual nodes; and
the resource quantity optimization module is further configured to: modify, after the simulation ends, the quantity of virtual resources of the virtual node to the set maximum resource quantity if the quantity of virtual resources of a certain virtual node is greater than a corresponding maximum resource quantity.

17. The optimization system according to claim 10, wherein the resource quantity optimization module further comprises:
a parameter setting unit, configured to determine a left boundary quantity N, a right boundary quantity 2*N, and a median M, where N is a current quantity of virtual resources of the node to be optimized, and M=(N+2*N)/2;
a resource quantity setting unit, configured to set the quantity of virtual resources of the node to be optimized to the current median M;
a resource quantity increasing unit, configured to set the quantity of virtual resources of the node to be optimized to M+1 if the current median M does not enable the quantity of virtual examination targets waiting to enter the node to be optimized to reach a minimum value;
a quantity resetting unit, configured to reset the left boundary quantity to M, and reset the median to M=(M+2*N)/2 if M+1 does not enable the quantity of the virtual examination targets waiting to enter the node to be optimized to reach the minimum value; otherwise, set the node to be optimized as an optimized node;
a resource quantity reducing unit, configured to set the quantity of virtual resources of the node to be optimized to M−1 if the current median M enables the quantity of the virtual examination targets waiting to enter the node to be optimized to reach the minimum value, wherein
the quantity resetting unit is further configured to: reset the right boundary quantity to M, and reset the median to M=(N+M)/2 if M−1 enables the quantity of the virtual examination targets waiting to enter the node to be optimized to reach the minimum value; otherwise, set the node to be optimized as an optimized node.

18. The optimization system according to claim 10, further comprising an analysis module configured to analyze one or both of an average waiting time and a resource utilization rate of each virtual node, wherein
the analyzing an average waiting time at each virtual node comprises: obtaining an average waiting time at each virtual node based on time information of each virtual examination target entering and exiting the virtual node; and
the analyzing a resource utilization rate of each virtual node comprises: analyzing, based on an idle time length of each virtual resource, a resource utilization rate of a virtual node where the virtual resource is located.

19. The optimization system according to claim 10, wherein the optimization system is disposed in a server, and the server is configured to communicate with one or a plurality of clients of a medical institution.

20. (canceled)

21. A non-transitory computer-readable storage medium, comprising a stored computer program, wherein the stored computer program, when executed by a processor, causes the processor to:
set a plurality of virtual examination targets and a plurality of virtual nodes, wherein the plurality of virtual nodes separately have an initial quantity of virtual resources; and
control the plurality of virtual examination targets to sequentially pass through the plurality of virtual nodes to simulate a plurality of actual examination phases in a medical examination procedure, wherein the simulating comprises determining a node to be optimized in the plurality of virtual nodes based on a current simulation result, and adjusting the quantity of virtual resources of the node to be optimized.
